(19) United States
(12) Patent Application Publication
(10) Pub. No.: US 2022/0040335 A1
(43) Pub. Date: Feb. 10, 2022

(54) RADIOLABELED PABA AND DERIVATIVES THEREOF FOR USE AS FUNCTIONAL RENAL IMAGING AGENTS

(71) Applicant: The Johns Hopkins University, Baltimore, MD (US)

(72) Inventors: Sanjay K. Jain, Baltimore, MD (US); Alvaro A. Ordonez, Baltimore, MD (US); Camilo A. Ruiz-Bedoya, Baltimore, MD (US)

(21) Appl. No.: 17/275,658

(22) PCT Filed: Sep. 11, 2019
(86) PCT No.: PCT/US2019/050529

§ 371 Date: Mar. 11, 2021

Related U.S. Application Data
(60) Provisional application No. 62/729,876, filed on Sep. 11, 2018.

(51) Int. Cl. A61K 51/04; C07B 59/00

(52) U.S. Cl. CPC A61K 51/0402; C07B 59/001

(57) ABSTRACT

The present invention provides positron emitter radiolabeled versions of PABA, metabolites and derivatives, with good radiochemical yield, high specific activity, high chemical and radiochemical purity and having excellent characteristics for PET imaging. The inventive composition and methods provide high quality dynamic images of the kidneys while reducing the radiation exposure. The short biological half-life of PABA, added to the short physical half-life of positron emitters such as ¹¹C will also benefit patients that require multiple renography assessments in a short period of time.

### REFERENCE TO RELATED APPLICATIONS

This application claims the benefit of U.S. Provisional Patent Application No. 62/729,876, filed on Sep. 11, 2018, and is hereby incorporated by reference for all purposes as if fully set forth herein.

### STATEMENT OF GOVERNMENTAL INTEREST

This invention was made with government support under grant nos. R01-EB020539 and R01-HL131829 awarded by the National Institutes of Health. The government has certain rights in the invention.

### BACKGROUND OF THE INVENTION

Para-aminobenzoic acid (4-aminobenzoic acid) (PABA) is a non-toxic B-complex vitamin substance with fast renal excretion, widely used since 1983 as an accepted exogenous objective marker to verify completeness of 24 hour urine sampling (1). PABA-verification is inter alia used to validate the accuracy of dietary intake studies (2), through the whole adult age range (3), often by calculating the association between dietary protein intake and urinary protein (nitrogen) excretion (4). The underlying assumption is that PABA is excreted almost quantitatively in 24 hours. While PABA is metabolized in the liver by phase II conjugation via N-acetyltransferase 1 and glycine conjugation, all its metabolites are also renally excreted (5, 6). In fact, one of its main metabolites, para-aminohippuric acid, has also been used to evaluate renal flow (7).

Accurate assessment of kidney function plays an essential role for optimal clinical decision making in a variety of diseases. Glomerular filtration rate (GFR) is considered as the most suitable index for assessing renal function and the gold standard for GFR estimation is through the determination of inulin clearance. However, its time-consuming nature and high cost limit its widespread use (8). Blood clearance using⁵¹Cr-ethylenediaminetetraacetic acid (⁵¹Cr-EDTA) is an alternative, but split renal function cannot be determined and imaging of the kidneys is not feasible (9). The filtered single-photon emission computed tomography (SPECT) agent ^{99m}Tc-diethylenetriaminepentaacetic acid (^{99m}Tc-DTPA) and ^{99m}Tc-mercaptuacetyltriglycine (^{99m}Tc-MAG3) are frequently used, in particular as it offers 3-dimensional imaging and anatomical co-registration with computed tomography (CT) (10), although these features are rarely used due to its prolonged acquisition and low single pass extraction which are seen as major obstacles for reliable dynamic imaging. On the contrary, the rapid 3-dimensional imaging capability of dynamic Positron Emission Tomography (PET)/CT not only overcomes this hurdle, but also provides superior spatio-temporal resolution, absolute quantification, and insights in cases of challenging anatomy (11).

There is still an unmet need for PET imaging tracers that provides GFR and rapid 3-dimensional imaging capability of the kidney to assess kidney structure and function.

### SUMMARY OF THE INVENTION

The present inventors show that radiolabeling PABA with positron emitting radionuclides such as ¹¹C, ¹³N, ¹⁸F, and others, can take advantage of the excellent characteristics of these radioisotopes for PET imaging, which provides high-quality dynamic images of the kidneys while reducing the radiation exposure from other radioisotopes. Therefore, the present inventors evaluated positron emitter labeled PABA could potentially be used to evaluate renal anatomy and function in a healthy rat model.

Recently, the present inventors developed a carbon 11-radiolabeled version of PABA with good radiochemical yield, high specific activity, high chemical and radiochemical purity (FIG. 1) (12). By radiolabeling PABA with ¹¹C, one can take advantage of the excellent characteristics of this radioisotope for PET imaging, which can provide high quality dynamic images of the kidneys while reducing the radiation exposure. The short biological half-life of PABA, added to the short physical half-life of most positron emitting radionuclides, such as ¹¹C, should also benefit patients that require multiple renography assessments in a short period of time.

Thus, in accordance with a plurality of embodiments, the present invention provides methods for functional renal imaging of one or both kidneys in a subject comprising administering to the subject an effective amount of PABA, or a metabolite or derivative thereof, labeled with a positron emitting radionuclide, obtaining a series of images at regular intervals of at least one or more regions of interest of the kidney for a set period of time, and quantifying the amount of radioactivity in each interval during the time period.

In accordance with an embodiment, the present invention provides a method for functional renal imaging one or both kidneys in a subject comprising administering to the subject an effective amount of one or more of the following compounds labeled with a positron emitting radionuclide: PABA, N-acetyl-PABA, and p-aminohippuric acid, or salts, solvates, and stereoisomers thereof, obtaining a series of images at regular intervals of at least one or more regions of interest of the kidney for a set period of time, and quantifying the amount of radioactivity in each interval during the time period.

In accordance with another embodiment, the present invention provides methods for functional renal imaging one or both kidneys in a subject comprising administering to the subject an effective amount of one or more of the following compounds labeled with ¹¹C:

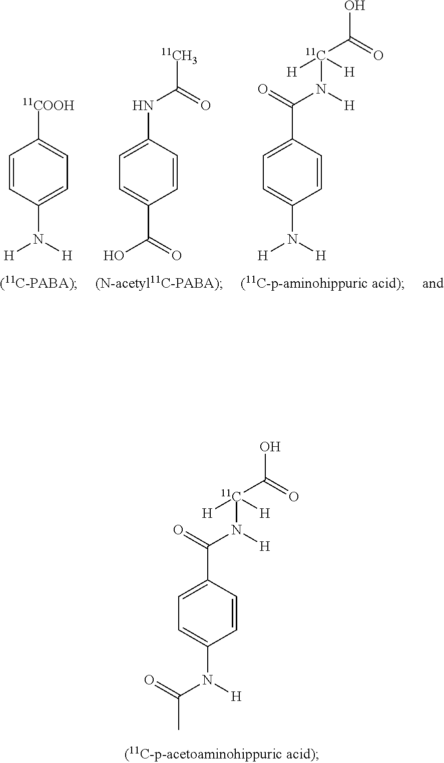

followed by obtaining a series of images at regular intervals of at least one or more regions of interest of the kidney for a set period of time, and quantifying the amount of radioactivity in each interval during the time period.

In accordance with another embodiment, the present invention provides methods for determining the kinetics of renal excretion of one or both kidneys in a subject comprising administering to the subject an effective amount of one or more of the following compounds labeled with a positron emitting radionuclide: PABA, N-acetyl-PABA, and p-aminohippuric acid, or salts, solvates, and stereoisomers thereof, obtaining a series of images at regular intervals of at least one or more regions of interest of the kidney for a set period of time, and quantifying the amount of radioactivity in each interval during the time period.

In accordance with another embodiment, the present invention provides a method for determining the kinetics of renal excretion one or both kidneys in a subject comprising administering to the subject an effective amount of one or more of the following compounds labeled with ¹¹C:

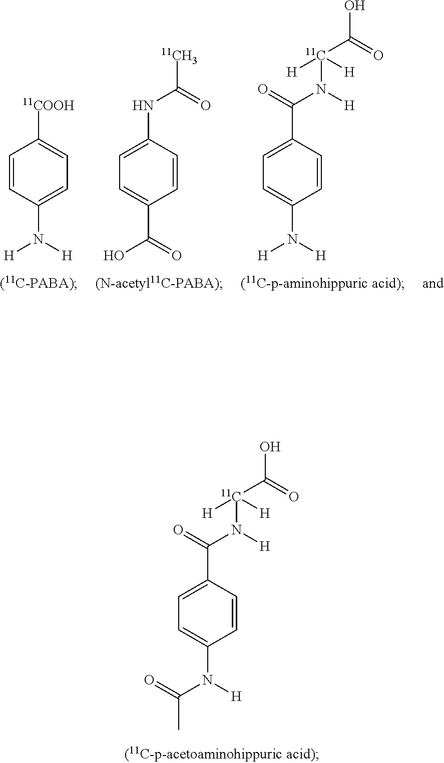

followed by obtaining a series of images at regular intervals of at least one or more regions of interest of the kidney for a set period of time, and quantifying the amount of radioactivity in each interval during the time period.

In accordance with another embodiment, the present invention provides methods for determining the renal structure of a region of interest of one or both kidneys in a subject comprising administering to the subject an effective amount of one or more of the following compounds labeled with a positron emitting radionuclide: PABA, N-acetyl-PABA, and p-aminohippuric acid, or salts, solvates, and stereoisomers thereof obtaining a series of images at regular intervals of at least one or more regions of interest of the kidney for a set period of time, and quantifying the amount of radioactivity in each interval during the time period.

In accordance with another embodiment, the present invention provides methods for determining the renal structure of a region of interest of one or both kidneys in a subject comprising administering to the subject an effective amount of one or more of the following compounds labeled with ¹¹C:

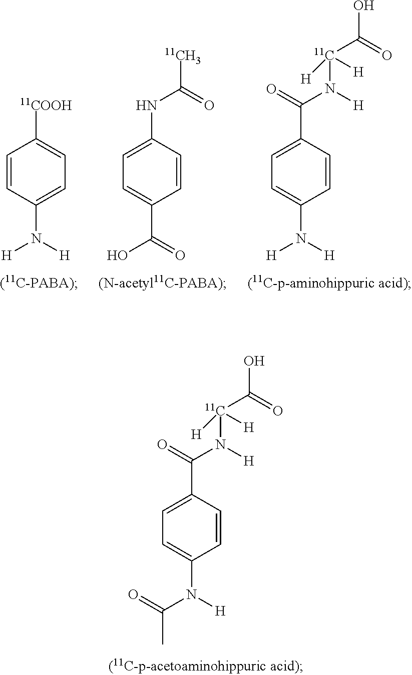

or salts, solvates, and stereoisomers thereof, obtaining a series of images at regular intervals of at least one or more regions of interest of the kidney for a set period of time, and quantifying the amount of radioactivity in each interval during the time period.

In accordance with an embodiment, the present invention provides a kit for use in a method for functional renal imaging and/or determining the kinetics of renal excretion of a kidney in a subject comprising a sufficient amount of PABA, or a metabolite or derivative thereof, labeled with a positron emitting radionuclide, along with any or all of the following: reagents, buffers, surgical implements, needles and implements for use in intravenous administration, sterile saline or another pharmaceutically acceptable emulsion and suspension base. In addition, the kits may include instructional materials, such as online continuing educational materials for training radiologists on how to interpret radiograms and containing directions (e.g., protocols) for the practice of the methods described herein.

### BRIEF DESCRIPTION OF THE DRAWINGS

FIG. 1 illustrates the radiosynthesis of ¹¹C-PABA. A cGMP-compliant synthesis of ¹¹C-PABA has been described by Holt et al. 2018 (8). The overall synthesis of the radiotracer product required approximately 15 minutes from end-of-bombardment. Subsequent QC testing adds another 15 minutes to the overall process. ¹¹C-PABA was obtained in good radiochemical yield, high specific activity, high chemical and radiochemical purity.

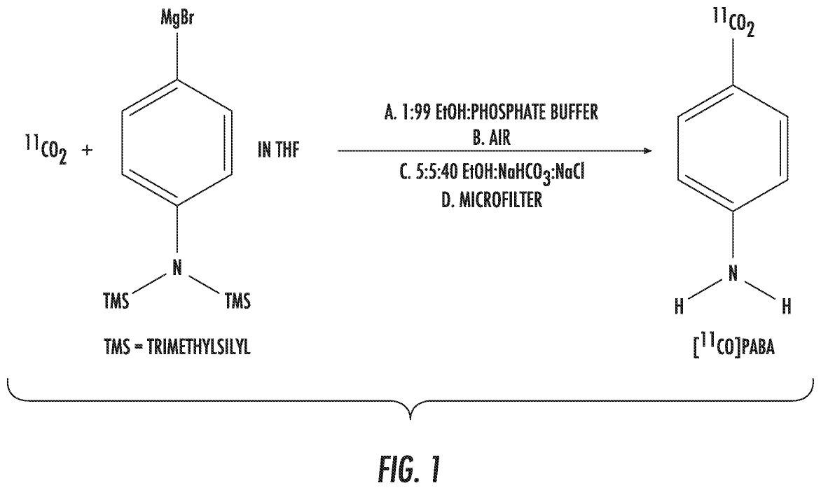

FIG. 2 shows ¹¹C-PABA renograms of healthy rats. Dynamic PET imaging after IV injection of ¹¹C-PABA showed rapid renal clearance with a time-to-peak in time-activity curves of 9.5±1.4 min. The Y-axis represents the percentage of the injected dose of radionuclide per cubic centimeter.

FIG. 3 depicts a ¹¹C-PABA renogram of a healthy rat and comparison with other organs. ¹¹C-PABA PET renogram from a representative rat showing the rapid vascular phase (0-4.7 min), followed by a cortical phase (4.7-10 min), and excretion phase which correlated with the increase of signal in bladder. The Y-axis represents the percentage of the injected dose of radionuclide per cubic centimeter.

FIG. 4A-4B shows that ¹¹C-PABA PET provides high quality anatomical images. (A) ¹¹C-PABA PET/CT signal localized in the right kidney 220-240 seconds after injection. (B) MIP reconstruction of ¹¹C-PABA PET/CT 10 minutes after injections shows the signal localized in the kidneys. The Y-axis represents the percentage of the injected dose of radionuclide per cubic centimeter.

### DETAILED DESCRIPTION OF THE INVENTION

In accordance with one or more embodiments, the present inventors provide novel methods for using PABA or a metabolite or derivative thereof, labeled with a positron emitting radionuclide for functional renal imaging. The use of these positron emitting radionuclide compounds can provide high-quality PET imaging of the kidney with a much lower level of radiation exposure to the tissues and subject.

Examples of radionuclides useful in accordance with the methods described herein include ¹¹C, ¹³N, ¹⁵O, ¹⁸F, ³⁸K, ⁴⁵Ti, ⁵¹Mn, ⁵²Mn, ⁵²Fe, ⁵⁵Co, ⁶⁰Cu, ⁶¹Cu, ⁶⁴Cu, ⁶⁶Ga, ⁷²As, ^{82m}Rb, ⁸³Sr, ⁸⁶Y, and ⁸⁹Zr.

Carbon-11 or ¹¹C is a radioactive isotope of carbon that decays to boron-11. This decay mainly occurs due to positron emission; however, around 0.19-0.23% of the time, it is a result of electron capture. It has a half-life of 20.334 minutes. Carbon-11 is commonly used as a radioisotope for the radioactive labeling of molecules in positron emission tomography. Among the many molecules used in this context are the radioligands 3-amino-4-(2-dimethylaminomethyl-phenylsulfanyl)benzonitrile [¹¹C]DASB and 2-(4-iodo-2,5-dimethoxyphenyl)-N-(2-[(11)C—OCH(3)]methoxybenzyl)ethanamine [¹¹C]Cimbi-5 for brain imaging for example.

PET is a nuclear, functional imaging technique that is used to evaluate metabolic processes within the body. The system is designed to detect pairs of gamma rays emitted indirectly by a positron-emitting radionuclide, which is introduced into the body on a biologically active molecule. This radionuclide is a tracer that highlights the metabolically active areas within organs and tissues. Tracers also provide information about blood flow and tissue oxygen consumption. Three-dimensional images of tracer concentration within the body are then constructed by computer analysis. The biologically active molecule often used for PET is ¹⁸F-fluorodeoxyglucose or (FDG), an analogue of glucose. Concentrations of FDG imaged typically indicate tissue metabolic activity as it corresponds to the regional glucose uptake by cells. As an example, PET scanning using FDG as the tracer is most commonly used and currently is a standard of care in the diagnosis and management of cancer.

The PET/CT scan has increased the ability of clinicians to diagnose and manage many medical conditions by melding precise anatomic localization with functional imaging. To this point, in current, the practice of medicine, the availability of PET/CT scan is rapidly changing the approach to pre-surgery mapping, radiation therapy, and cancer staging. As a result, many centers are gradually abandoning conventional PET devices and substituting them by PET-CT scanners.

It will be understood by those of ordinary skill in the art that the methods of the present invention are directed generally to the use of PABA, or a metabolite or derivative thereof, labeled with a positron emitting radionuclide, in performance of radioisotope renography or renal scintigraphy. Radioisotope renography provides important functional data to assist in the diagnosis and management of patients with a variety of suspected genitourinary tract problems.

Thus, in accordance with a plurality of embodiments, the present invention provides methods for functional renal imaging of one or both kidneys in a subject comprising administering to the subject an effective amount of PABA, or a metabolite or derivative thereof, labeled with a positron emitting radionuclide, obtaining a series of images at regular intervals of at least one or more regions of interest of the kidney for a set period of time, and quantifying the amount of radioactivity in each interval during the time period.

As used herein, the terms “functional renal imaging” and “radioisotope renography” or “renal scintigraphy” are synonymous and comprise intravenous administration of subject an effective amount of PABA, or a metabolite or derivative thereof, labeled with a positron emitting radionuclide for assessment of renal function and anatomy, including, for example, diuresis renography, captopril augmented renography, and renal transplant.

As used herein, the term “of PABA, or a metabolite or derivative thereof,” means the compound 4-aminobenzoic acid, its metabolites, such as, N-acetyl-PABA and 4-parahippouric acid. In addition, derivatives of PABA, such as C₁-C₃ alkyl, OH, amino, carboxyl, C₁-C₃ alkylamino, and halo are contemplated.

In some embodiments, the present invention provides methods for functional renal imaging one or both kidneys in a subject comprising administering to the subject an effective amount of one or more of the following compounds labeled with a positron emitting radionuclide: PABA, N-acetyl-PABA, and p-aminohippuric acid, or salts, solvates, and stereoisomers thereof, obtaining a series of images at regular intervals of at least one or more regions of interest of the kidney for a set period of time, and quantifying the amount of radioactivity in each interval during the time period.

In accordance with another embodiment, the present invention provides methods for functional renal imaging one or both kidneys in a subject comprising administering to the subject an effective amount of one or more of the following compounds labeled with ¹¹C:

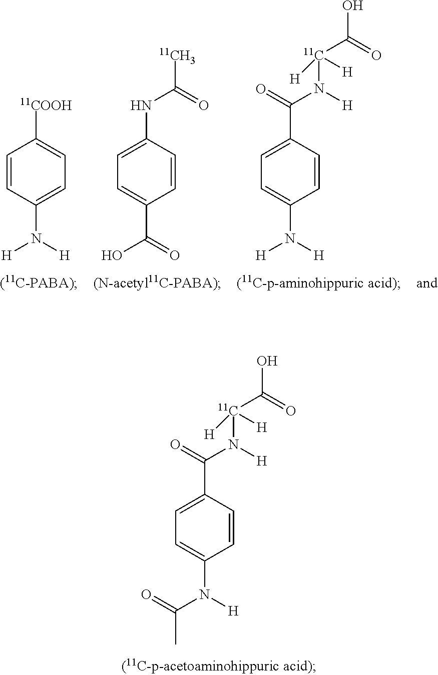

followed by obtaining a series of images at regular intervals of at least one or more regions of interest of the kidney for a set period of time, and quantifying the amount of radioactivity in each interval during the time period.

Generally speaking, renal scans used in functional renal imaging are frequently performed after the intravenous injection of approximately 370 MBq (10 mCi) of PABA, or a metabolite or derivative thereof. Administration of activities in the range of 370 MBq may be required to obtain sufficient counts to visualize the initial bolus as it transits the aorta and kidneys or to calculate quantitative flow indices, however, except for the evaluation of renal transplants, neither 2-s flow images nor quantitative flow calculations obtained in the first few seconds after injection have been clearly demonstrated to contribute to the evaluation of relative function, suspected obstruction, or renovascular hypertension. Images are acquired dynamically for 20-30 min in 10- to 20-s frames and are usually displayed at 1- or 2-min intervals as the radioactive tracer is removed from the blood, transits the kidney, and enters the bladder. Postvoid views of the kidneys and bladder are recommended at the conclusion of the study. The degree of extravasation or infiltration can be estimated by dividing the counts/min at the site of infiltration by the counts/min injected.

Time-activity curves are generated after placement of a region of interest (ROI) over each kidney, the renal cortex (parenchyma), or retained activity in the collecting system. Data are recorded on a computer or data storage device for subsequent analysis. The whole-kidney ROI consists of an ROI placed around the entire kidney, including the renal pelvis. Quantitative values generated using this ROI will be affected by retention of tracer in both the kidney parenchyma and the renal pelvis. Tracer retention may occur in pathologic states such as diabetic nephropathy or obstruction but may also occur in nonpathologic states such as a nonobstructed dilated collecting system or mild dehydration. To obtain a better assessment of parenchymal function, ROIs can be restricted to the renal cortex (parenchyma), excluding any retained activity in the renal pelvis or calyces.

Time-Activity (Renogram) Curves. Whole kidney and cortical (parenchymal) time-activity curves are illustrated typically as the difference in the relative height of the cortical curves is due to the differences in the size of the relative cortical ROIs. The function of the cortical curve is to display the transit time through the cortex without contaminating the curve from activity in the collecting system; the cortical (parenchymal) ROIs are not drawn to have equal areas but to exclude the renal pelvis. Unless the cortical counts are normalized for area, a cortical ROI with a larger area will have more counts than a contralateral cortical ROI with a smaller area; consequently, the renogram curve with the larger cortical ROI will have a greater maximum. It is the shape of the cortical renogram curve that is important, not the absolute height. It is important to note that parameters generated from the cortical renogram curves may be spurious because of patient motion; parameters generated from cortical curves derived from poorly functioning kidneys may also be spurious because of low counts and noise.

Quantitative Indices.

Relative perfusion. Renal perfusion is evaluated by the visual or quantitative analysis of the initial bolus as it transits the abdominal aorta and enters the renal arteries. With the exception of renal transplant evaluation, quantitative measurements of renal perfusion have not been demonstrated to contribute to renal scan interpretation and can be omitted.

Relative Function. The relative uptake of the radiopharmaceutical provides a measure of differential renal function (the specific function depends on the radiopharmaceutical) and should be reported. For the PABA derivative used in the methods of the present invention, the measurement can be made by placing an ROI over each kidney and measuring the integral of the counts in renal ROI during the 1- to 2-, 1- to 2.5-, or 2- to 3-min period after injection or using the Rutland-Patlak plot. With the integral approach, the measurement needs to be made before any tracer is eliminated by either kidney. In patients with bilaterally impaired function and delayed excretion, the kidney-to-background ratio will be reduced compared with normally functioning kidneys; in this setting, the differential function measurement will be more accurate if the measurement is obtained not during the 1- to 3-min post-injection period but rather in the 1-min period just before any tracer leaves the kidney ROI.

Renal size. Knowledge of renal size can assist in the interpretation of renal scans. Several chronic renal diseases will result in bilaterally small kidneys, whereas the kidneys may be bilaterally enlarged in early diabetic renal disease, acute interstitial nephritis, HIV nephropathy, and amyloidosis. A unilaterally small kidney is obvious, but a small kidney may not be obvious on a renal scan when the contralateral kidney is similarly reduced in size; conversely, an abnormal increase in renal size may not be recognized if both kidneys are similarly enlarged but have a normal configuration. Correlative imaging studies may provide size information, but unpublished results from our institution indicate that approximately 50% of patients referred for renal scintigraphy lack prior imaging studies; moreover, even when reports are available, there is often no comment regarding renal size. Renal size (length in cm and area in cm²) can be determined from the pixel length and area of the whole-kidney ROT Regression equations to define the upper and lower limits of renal size normalized for body surface area have recently been developed for adults. Routine measurement of renal size at the time of the scan may assist in the detection of unsuspected bilateral increases or decreases in renal size and facilitate scan interpretation.

Time to peak or Tmax. The time to peak, or Tmax, simply refers to the time from radiopharmaceutical injection to the peak height of the renogram curve. The physiologic retention of the tracer in the renal calyces or pelvis can alter the shape of the whole-kidney renogram curve in normal kidneys and lead to prolonged values for the time to peak, 20-min/maximum count ratio, and T½. The T½ refers to the time it takes for the activity in the kidney to fall to 50% of its maximum value. The 20-min/maximum count ratio is the ratio of the kidney counts at 20 min to the maximum (peak) counts; this measurement provides an index of the transit time and parenchymal function and is often obtained for both whole kidney and cortical (parenchymal) ROIs. If the patient is not dehydrated and the 20-min/maximum count ratio for the cortical ROI exceeds 0.35 (greater than 2-3 SDs above the mean), the kidney is likely to be abnormal. In addition to detecting abnormal function, the 20-min/maximum and 20-min/1- to 2-min count ratios can be useful in monitoring patients with suspected urinary tract obstruction and renovascular hypertension

The radiotracer is filtered by the glomeruli without reabsorption by the renal tubules. Dynamic PET can determine the concentration of the radiotracer (¹¹C-PABA or ^{99m}Tc-DTPA) in the kidneys and blood to calculate GFR. Blood and urine samples are also usually taken to calculate excretion rate of the radiotracer determined by gamma counting. The valuable and accurate information provided by the renogram can help clinicians to diagnose urinary system obstruction and monitor transplanted kidneys and the effects of treatment and operation.

Glomerular filtration rate (GFR). Typically a non-imaging blood test for GFR measurement is made using Tc-99m DTPA. Serial venous samples are obtained at different time points over a 3 or 4 hour period following injection. Plasma is obtained and radioactivity counted in a well counter to quantify the clearance of the tracer over time. An exponential model is fitted to the data and GFR is determined from the clearance rate.

In accordance with an embodiment, the present invention provides a kit for use in a method for functional renal imaging of one or both kidneys in a subject comprising a sufficient amount of positron labeled-para aminobenzoic acid, along with any or all of the following: reagents, buffers, surgical implements, needles and implements for use in intravenous administration, sterile saline or another pharmaceutically acceptable emulsion and suspension base. In addition, the kits may include instructional materials, such as online continuing educational materials for training radiologists on how to interpret radiograms and containing directions (e.g., protocols) for the practice of the methods described herein.

To scan the kidney for functional diagnosis, tumors or neoplasias, a radiopharmaceutical preparation in accord with this invention having a suitable dose of radioactivity for the particular subject is contained in a suitable pharmacological carrier such as normal saline. The radiopharmaceutical preparation is injected intravenously into the mammal. The kidney is then imaged by positioning the mammal under a PET camera in such a way that the kidney is covered by the field of view.

In accordance with an embodiment, the present invention provides a method for functional renal imaging of one or both kidneys in a subject comprising: a) administering to the subject an effective amount of positron labeled PABA, or a metabolite or derivative thereof; b) imaging the kidney of the subject with a positron emission detecting apparatus or PET or SPECT device; c) collecting the data on a computer or data storage device; d) identifying a ROI and analyzing the data of c).

In some embodiments, the method above can further include the step of e) comparing the analyzed images of d) of the subject with analyzed images of a control kidney or kidneys.

In some embodiments, the method can further comprise a step of graphing the data as time-activity curve.

In some embodiments of the inventive methods, at step b), the imaging time is between about 5 minutes, to about 60 minutes, in some embodiments, from about 5 minutes to about 40 minutes.

In some embodiments, the images are acquired dynamically for a period of time in 10- to 20-s frames.

In some embodiments of the inventive methods, at step d), the analysis can be visualized, or calculated with a computer. The computer can be any digital storage means and can be different than the computer or instrument which collects the imaging data of the methods.

As used herein, the functional renal imaging methods disclosed can be useful for a wide variety of diagnostic and therapeutic uses. The methods herein can detect changes in blood flow, such as blockages or occlusions, the causes of which include, but are not limited to congenital malformations, metabolic disorders, neoplasias, infections, inflammation, and injury.

In accordance with an embodiment, the present invention provides an imaging agent for use in functional renal imaging of one or both kidneys in a subject in need thereof, characterized in that the imaging agent comprises para-amino benzoic acid (PABA) or a metabolite or derivative thereof, labeled with a positron emitting radionuclide, and wherein the imaging agent is administered to the subject in conjunction with obtaining a series of images at regular intervals of at least one or more regions of interest of the kidney of the subject for a period of time, and quantifying the amount of radioactivity in each interval during said time period.

In some embodiments, the imaging agent is labeled with a positron emitting radionuclide selected from the group consisting of: ¹¹C, ¹³N, ¹⁵O, ¹⁸F, ³⁸K ⁴⁵Ti, ⁵¹Mn, ⁵²Mn, ⁵²Fe, ⁵⁵Co, ⁶⁰Cu, ⁶¹Cu, ⁶⁴Cu, ⁶⁶Ga, ⁷²As, ^{82m}Rb, ⁸³Sr, ⁸⁶Y, and ⁸⁹Zr.

In some embodiments, the PABA metabolite is selected from the group consisting of N-acetyl-PABA, p-aminohippuric acid, and p-acetylaminohippuric acid.

In accordance with an embodiment, the present invention provides an imaging agent for use in functional renal imaging of one or both kidneys in a subject in need thereof, characterized in that the imaging agent comprises a compound selected from the group consisting of:

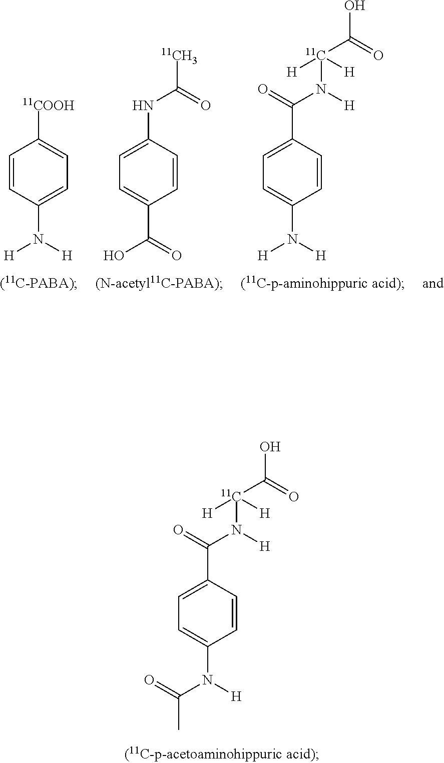

and wherein the imaging agent is administered to the subject in conjunction with obtaining a series of images at regular intervals of at least one or more regions of interest of the kidney of the subject for a period of time, and quantifying the amount of radioactivity in each interval during said time period.

In accordance with an embodiment, the present invention provide an imaging agent for use in functional renal imaging of one or both kidneys in a subject in need thereof, characterized in that the imaging agent comprises para-amino benzoic acid (PABA) or a metabolite or derivative thereof, labeled with a positron emitting radionuclide, and wherein the imaging agent is administered to the subject in conjunction with imaging the kidney of the subject with a positron emission detecting apparatus or PET or SPECT device at regular intervals of at least one or more regions of interest of the kidney of the subject for a period of time, quantifying the amount of radioactivity in each interval during said time period, collecting the quantifying data on a computer or data storage device; and identifying a ROI and analyzing the data of the images collected.

In some embodiments, the analyzed images of the subject are compared with analyzed images of a control kidney or kidneys.

In some embodiments, the data of the images collected is graphed as time-activity curve.

In some embodiments, the imaging agent is administered to the subject along with the administration to the subject of a second positron labeled imaging agent.

In some embodiments, the imaging agent is administered to the subject along with a pharmacological agent prior to or concurrently with the imaging agent.

In some embodiments, the pharmacological agent is selected from the group consisting of. Captopril, Enalapril, Aspirin, ARA II (valsartan) and other angiotensin II inhibitors, and furosemide and other diuretics.

Common clinical indications for use of the methods disclosed herein include, but are not limited to acute and chronic renal failure, unilateral and bilateral renal disease, obstructive uropathy, renovascular hypertension, status post-renal transplantation, pyelonephritis and parenchymal scarring.

In accordance with some embodiments, the positron labeled PABA compounds, metabolites and derivatives are used with other pharmacological agents to further assess the structure and functioning of the kidney or kidneys in a subject. Examples of such drugs which can be administered to a subject prior to, or during the renogram procedure include, but are not limited to, Captopril (Captoptril radionuclide renography)—used to evaluate the Renin-Angiotensin-Aldosterone system, Enalapril, Aspirin, ARA II (valsartan) and others of the class of angiotensin II inhibitors, and furosemide and others of the class of diuretics.

It will also be understood that other known tracers can be used in conjuction with the PABA metabolites and derivatives disclosed herein, including, for example, ^{99m}Tc MAG3, ^{99m}Tc-EC (ethylenecysteine), and ^{99m}Tc-DTPA (diethylenetriamine pentaacetic acid), among others.

A pharmacological agent and a biologically active agent are used interchangeably herein to refer to a chemical or biological compound that induces a desired pharmacological and/or physiological effect, wherein the effect may be prophylactic or therapeutic. The terms also encompass pharmaceutically acceptable, pharmacologically active derivatives of those active agents specifically mentioned herein, including, but not limited to, salts, esters, amides, prodrugs, active metabolites, analogs and the like. When the terms “active agent,” “pharmacological agent” and “drug” are used, then, it is to be understood that the invention includes the active agent per se as well as pharmaceutically acceptable, pharmacologically active salts, esters, amides, prodrugs, metabolites, analogs etc.

“Treating” or “treatment” is an art-recognized term which includes curing as well as ameliorating at least one symptom of any condition or disease. Treating includes reducing the likelihood of a disease, disorder or condition from occurring in an animal which may be predisposed to the disease, disorder and/or condition but has not yet been diagnosed as having it; inhibiting the disease, disorder or condition, e.g., impeding its progress; and relieving the disease, disorder or condition, e.g., causing any level of regression of the disease; inhibiting the disease, disorder or condition, e.g., impeding its progress; and relieving the disease, disorder or condition, even if the underlying pathophysiology is not affected or other symptoms remain at the same level.

“Prophylactic” or “therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

Injectable formulations are in accordance with the invention. The requirements for effective pharmaceutical carriers for injectable compositions are well-known to those of ordinary skill in the art (see, e.g., Pharmaceutics and Pharmacy Practice, J. B. Lippincott Company, Philadelphia, Pa., Banker and Chalmers, eds., pages 238-250 (1982), and ASHP Handbook on Injectable Drugs, Trissel, 15th ed., pages 622-630 (2009)).

The compositions of the present invention may include a carrier. The term, “carrier,” refers to a diluent, adjuvant, excipient or vehicle with which the therapeutic is administered. Such physiological carriers can be sterile liquids, such as water. Water is a suitable carrier when the pharmaceutical composition is administered intravenously. Saline solutions and aqueous dextrose and glycerol solutions also can be employed as liquid carriers, particularly for injectable solutions. The compositions, if desired, can also contain minor amounts of wetting or emulsifying agents, or pH buffering agents.

Buffering agents help to maintain the pH in the range which approximates physiological conditions. Buffers are preferably present at a concentration ranging from about 2 mM to about 50 mM. Suitable buffering agents for use with the instant invention include both organic and inorganic acids, and salts thereof, such as citrate buffers (e.g., monosodium citrate-disodium citrate mixture, citric acid-trisodium citrate mixture, citric acid-monosodium citrate mixture etc.), succinate buffers (e.g., succinic acid monosodium succinate mixture, succinic acid-sodium hydroxide mixture, succinic acid-disodium succinate mixture etc.), tartrate buffers (e.g., tartaric acid-sodium tartrate mixture, tartaric acid-potassium tartrate mixture, tartaric acid-sodium hydroxide mixture etc.), fumarate buffers (e.g., fumaric acid-monosodium fumarate mixture, fumaric acid-disodium fumarate mixture, monosodium fumarate-disodium fumarate mixture etc.), gluconate buffers (e.g., gluconic acid-sodium glyconate mixture, gluconic acid-sodium hydroxide mixture, gluconic acid-potassium gluconate mixture etc.), oxalate buffers (e.g., oxalic acid-sodium oxalate mixture, oxalic acid-sodium hydroxide mixture, oxalic acid-potassium oxalate mixture etc.), lactate buffers (e.g., lactic acid-sodium lactate mixture, lactic acid-sodium hydroxide mixture, lactic acid-potassium lactate mixture etc.) and acetate buffers (e.g., acetic acid-sodium acetate mixture, acetic acid-sodium hydroxide mixture etc.). Phosphate buffers, carbonate buffers, histidine buffers, trimethylamine salts, such as Tris, HEPES and other such known buffers can be used.

The dose of the PABA, metabolites and derivatives of the compositions used in the methods of the present invention also will be determined by the existence, nature and extent of any adverse side effects that might accompany the administration of a particular compound. Typically, an attending physician will decide the dosage of the compound with which to administer to each individual patient, taking into consideration a variety of factors, such as age, body weight, general health, diet, sex, compound to be administered, and route of administration. By way of example, and not intending to limit the invention, the dose of the compounds of the present invention should achieve kidney levels suitable for PET or SPECT or other imaging. In accordance with some embodiments, the dose range for the agent is in the range of about 37-370 MBq of ¹¹C-PABA, metabolites and derivatives. Preferably, the dose range for the agent is about 37-370 MBq of ¹¹C-PABA.

As used herein, the term “subject” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

In accordance with some other embodiments, educational materials, including online and computer-based teaching tools which allow training of radiologists in the use of the diagnostic methods described herein, are also contemplated. Such tools would include manuals with reference images, clinical protocols and other materials known to those of skill in the art.

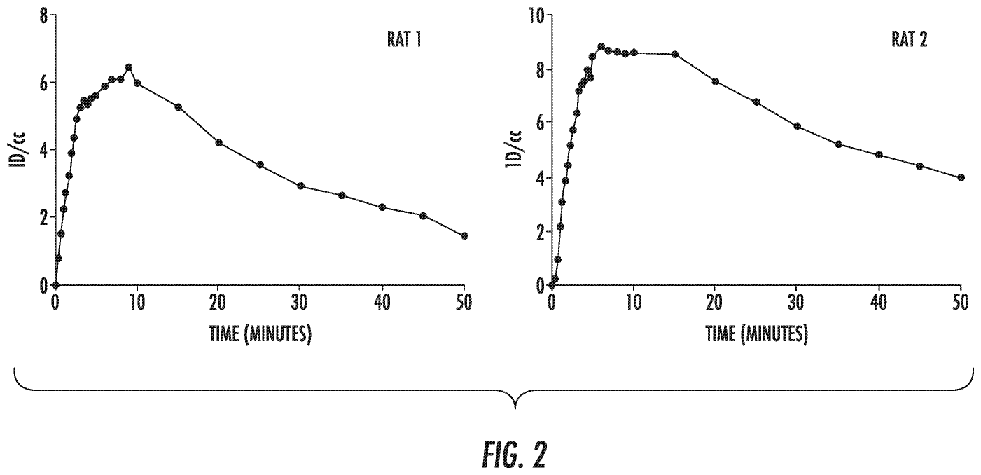

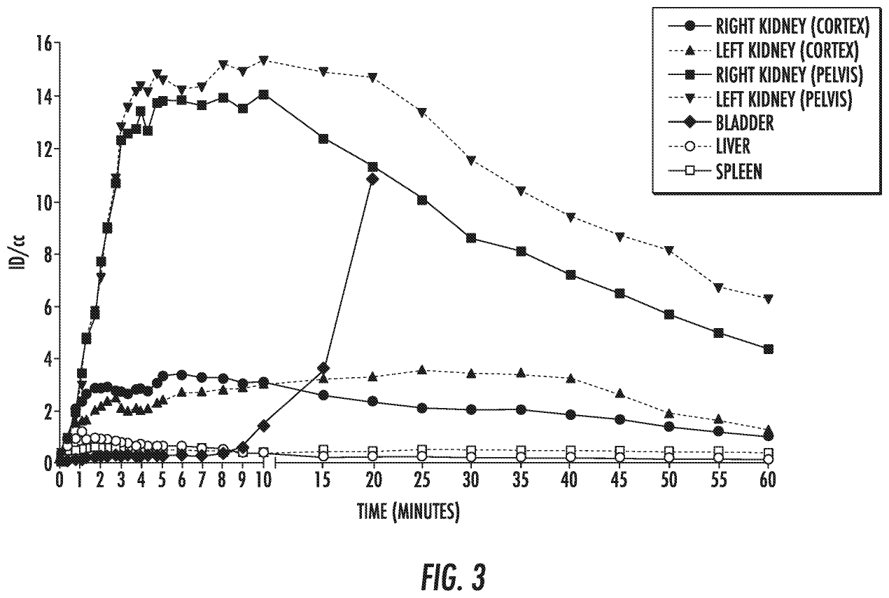

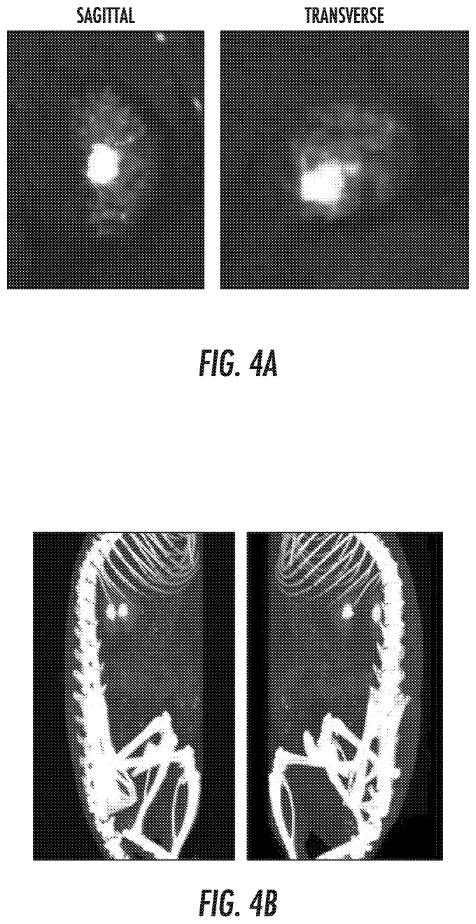

By doing dynamic PET imaging using the inventive methods disclosed herein, the inventors determined the kinetics of renal excretion of ¹¹C-PABA after an intravenous injection in healthy rats. The tracer rapidly localized in the kidneys (FIG. 2). ¹¹C-PABA showed a rapid vascular phase, followed by a cortical phase and excretion phase, which correlated with the increase of signal in the bladder (FIG. 3). Overall, the background signal was very low. Due to the advantages of PET imaging, ¹¹C-PABA PET was able to provide three-dimensional detailed images of the renal anatomy (FIG. 4).

The following examples have been included to provide guidance to one of ordinary skill in the art for practicing representative embodiments of the presently disclosed subject matter. In light of the present disclosure and the general level of skill in the art, those of skill can appreciate that the following examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### EXAMPLES

Methods for renal imaging: ¹¹C-PABA was synthesized using cGMP conditions at the JHU PET Center. Four female healthy Wistar rats (250-275 g of bodyweight) were injected with 30.5±10.3 MBq of ¹¹C-PABA through a tail-vein catheter and a dynamic PET scan was performed (15 frames×20 seconds, 5 frames×1 minute, 10 frames×5 minutes, total of 60 minutes) using the nanoScan PET/CT (Mediso). Subsequently, CT imaging was performed for anatomical co-registration. As a control, the same rats were also injected intravenously with 18.5 MBq of ^{99m}Tc-MAG3 (Cardinal Health) and scanned using planar scintigraphy with the nanoSPECT/CT (Nanoscan). The images were analyzed with VivoQuant 3.0 (Invicro) and regions of interest were drawn using the CT as reference.

Synthesis of 2-[¹⁸F]-F-PABA.

Synthesis of 2-[¹⁸F]-F-PABA was done according to the following scheme:

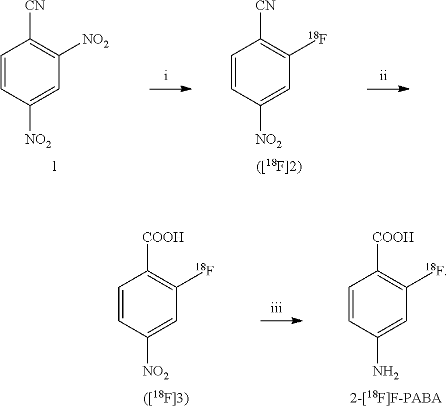

Reagents and conditions: (i) [¹⁸F]potassium fluoride, Kryptofix222, potassium carbonate, dimethyl sulfoxide, r.t., 10 min; (ii) 2M potassium hydroxide in water, 105° C., 10 min; (iii) zinc powder, ammonium chloride, water, 105° C., 5 min. Overall decay-corrected radiochemical yield: 30-40% (n=3). The radioactive product was characterized by radio-HPLC which determined the chemical and radiochemical impurities in the product and compared the retention time of the labeled material to a cold reference standard. The overall synthesis time was 90 min with a typical overall decay corrected radiochemical yield of 30-40%. 2-[¹⁸F]F-PABA had a specific activity of 240.5±77.7 GBq/μmole (6.5±2.1 Ci/μmole, n=4) and a radiochemical purity of 99.2±0.7% (n=4). We utilized a conventional nucleophilic aromatic substitution (SNAr) reaction for radiofluorination. The precursor, 2,4-dinitrobenzonitrile (1), is commercially available and is readily radiofluorinated to give intermediate [¹⁸F]. Basic hydrolysis of the nitrile followed by reduction of the 4-nitro moiety proceeded rapidly to produce 2-[¹⁸F]F-PABA after purification by radio-HPLC. The entire radiosynthesis was automated using a GE Tracerlab FXN Pro Radiosynthesis Module which should facilitate the translation of 2-[¹⁸F]F-PABA to clinical applications.

### Example 1

Radiosynthesis of ¹¹C-PABA. A cGMP-compliant synthesis of ¹¹C-PABA has been described by Holt et al. 2018 (8). The overall synthesis of the radiotracer product required approx. 15 minutes from end-of-bombardment. Subsequent QC testing adds another 15 minutes to the overall process. ¹¹C-PABA was obtained in good radiochemical yield, high specific activity, high chemical and radiochemical purity (adapted from Holt et al. 2018).

Synthesis of ¹¹C-PABA:

Production of [¹¹C] carbon dioxide: Pressurized ultra-high purity nitrogen gas (99.5%) mixed with 0.5% oxygen (Roberts Oxygen, Baltimore, Md.) in a standard carbon-11 PETtrace target (General Electric Medical Systems (GEMS), Waukesha, Wis.) was irradiated with a 16 MeV proton beam of 60 pA for up to 30 minutes to produce in approximately 1.5 Ci (37 GBq) of [¹¹C] carbon dioxide via the ¹⁴N(p,α)¹¹C nuclear reaction.

Concentration and purification of [¹¹C] carbon dioxide: Prior to their use, a molecular sieve trap (Alltech, 13X; approximately 0.4 g) and a Carbosphere packed GC column (⅛″×6′ stainless steel column packed with Carbosphere 60/80; Alltech) heated at 325° C. and 150° C., respectively, for 5 minutes each while flowing nitrogen gas (UHP; 99.9995%) at approximately 100 mL/min. Following the cyclotron bombardment, the target output of [¹¹C]CO₂ was concentrated on the molecular sieve trap for 120 seconds, while other radioactive gases were diverted to waste. The molecular sieve was heated for 130 seconds from ambient room temperature to a maximum temperature of 325° C., resulting in the transfer of [¹¹C]CO₂ at approximately 85 mL/min in a stream of nitrogen (99.999%; Roberts Oxygen) onto the Carbosphere packed GC column at ambient room temperature. The Carbosphere column was then heated for 195 seconds to a maximum temperature of 150° C. while the purified [¹¹C]CO₂ was flushed to the next step of the reaction.

Reaction with Grignard solution: Prior to its use, a 2-mL reaction loop of ⅛″ ID fluorinated ethylene propylene tubing was sequentially washed with 5 mL aliquots of hydrochloric acid (2 M), HPLC water, and acetone, after which the loop was flushed with nitrogen gas (UHP, 99.9995%) for at least 15 minutes (100 mL/min). Purified [¹¹C]CO₂ from the previous step was flushed through the loop that was prefilled with 4-[bis (trimethylsilyl)amino] phenylmagnesium bromide in tetrahydrofuran (THF) (100 μL, 0.5 M; Sigma-Aldrich, St. Louis, Mo.) mixed with THF (100 μL, Sigma-Aldrich). Trapping of the [¹¹C]CO₂ in the Grignard loop took approximately 40 seconds.

Purification of the radiotracer product: Using a remotely controlled, multiport Cavro pump (Tecan, Morrisville, N.C.), a mixture (8 mL) of 1:99 Dehydrated Alcohol USP:0.75 M phosphate buffer pH 2.2 solution (Dehydrated Alcohol USP, Pharmco-Aaper, Shelbyville, Ky., and sodium dihydrogen phosphate monohydrate and phosphoric acid for the buffer solution, Sigma-Aldrich) was passed through the reaction loop, a syringe filter (to trap particulates; Millipore-Sigma, Millex 0.45 μm LCR filter, 25 mm), and an Oasis HLB Sep-Pak Plus (Waters Corp, Milford, Mass.) to waste (note: Flow path dead volume through the loop and syringe filter intentionally results in 4 mL of solution from passing through the Sep-Pak). Next, the pump washed an additional 7 mL of 1:99 Dehydrated Alcohol USP: 0.75 M phosphate buffer pH 2.2 solution through just the Oasis HLB Sep-Pak Plus alone to waste. Then, the pump passed 7 mL of Sterile Water for Injection (Hospira, Lake Forest, New Jersey) through the Oasis HLB Sep-Pak Plus to waste, followed by 5 mL of filtered lab air. The [¹¹C] PABA radiotracer product was eluted from the Oasis HLB Sep-Pak Plus with 10 mL of 10:10:80 dehydrated alcohol USP: 8.4% sodium bicarbonate Inj: sodium chloride, 0.9% injection (sodium bicarbonate and sodium chloride obtained from Hospira) through the Millipore FG sterile filter assembly (Millipore-Sigma) into a sterile, pyrogen-free vial (NUCMEDCOR, San Francisco, Calif.) prefilled with 4 mL of sodium chloride, 0.9% Injection. The final product vial was immediately transferred from the production area to the quality control (QC) laboratory for analysis.

### Example 2

In vivo PET imaging visualized rapid excretion of ¹¹C-PABA from both kidneys, with very low background accumulation of the tracer in other organs (FIG. 2). Initial cortical tracer uptake followed by visualization of the collecting system could be observed. For ¹¹C-PABA, the time-to-peak in time-activity curves was 9.5±1.4 min. At this time point, the ¹¹C-PABA signal in the kidneys was 30-40× higher compared to liver and spleen (FIG. 3). ¹¹C-PABA PET/CT signal localized in the right kidney 220-240 seconds after injection (FIG. 4A). MIP reconstruction of ¹¹C-PABA PET/CT 10 minutes after injections shows the signal localized in the kidneys (FIG. 4B).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### REFERENCES

1. Bingham, S & Cummings, JH (1983). The use of 4-aminobenzoic acid as a marker to validate the completeness of 24 h urine collections in man. Clin. Sci., 64, 629-635.
2. Bingham, S A, Cassidy, A, Cole, T J, Welch, A, Runswick, S A, Black, A E, Thurnham, D, Bates, C, Khaw, K T, Key, T J A & Day, NE (1995). Validation of weighed records and other methods of dietary assessment using the 24 h urine nitrogen technique and other biological markers. Br. J. Nutr., 73, 531-550.
3. Black, A E, Bingham, S A, Johansson, G & Coward, W A (1997). Validation of dietary intakes of protein and energy against 24 hour urinary N and DLW energy expenditure in middle-aged women, retired men and post-obese subjects: comparisons with validation against presumed energy requirements. Eur. J. Clin. Nutr., 51, 405-413.
4. Bingham, S & Cummings, JH (1985). Urine nitrogen as an independent validatory measure of dietary intake: a study of nitrogen balance in individuals consuming their normal diet. Am. J. Clin. Nutr., 42, 1276-1289.
5. Lebel S, Nakamachi Y, Hemming A, Verjee Z, Phillips M J, Furuya K N. Glycine conjugation of para-aminobenzoic acid (PABA): a pilot study of a novel prognostic test in acute liver failure in children. Journal of pediatric gastroenterology and nutrition. 2003; 36(1):62-71.
6. Tabor C W, Freeman M V, Baily J, SMITH P. Studies on the metabolism of para-aminobenzoic acid. Journal of Pharmacology and Experimental Therapeutics. 1951; 102:98-102.
7. Setcchell B, Blanch E. Conjugation of p-aminohippurate by the kidney and effective renal plasma-flow. Nature. 1961; 189(4760):230.
8. Holt D P, Kalinda A S, Bambarger L E, Jain S K, Dannals R F. Radiosynthesis and Validation of [Carboxy-11C] 4-Aminobenzoic acid ([11C] PABA), A PET Radiotracer for Imaging Bacterial Infections. Journal of Labelled Compounds and Radiopharmaceuticals. 2018
9. Chantler C, Garnett E S, Parsons V, Veall N. Glomerular filtration rate measurement in man by the single injection methods using 51Cr-EDTA. Clin Sci. 1969; 37(1):169-80.
10. Hofman M S, Hicks R J. Gallium-68 EDTA PET/CT for Renal Imaging. Semin Nucl Med. 2016; 46(5):448-61.
11. Hofman M, Binns D, Johnston V, Siva S, Thompson M, Eu P, et al. 68Ga-EDTA PET/CT imaging and plasma clearance for glomerular filtration rate quantification: comparison to conventional 51Cr-EDTA. J Nucl Med. 2015; 56(3):405-9.
12. Holt D P, Kalinda A S, Bambarger L E, Jain S K, and Dannals R F. J Labelled Comp Radiopharm. 2018 Aug. 8. doi: 10.1002/jlcr.3674.

1. An imaging agent for use in functional renal imaging of one or both kidneys in a subject in need thereof, characterized in that the imaging agent comprises para-amino benzoic acid (PABA) or a metabolite or derivative thereof, labeled with a positron emitting radionuclide, and wherein the imaging agent is administered to the subject in conjunction with obtaining a series of images at regular intervals of at least one or more regions of interest of the kidney of the subject for a period of time, and quantifying the amount of radioactivity in each interval during said time period.

2. An imaging agent for use as in claim 1, wherein the a positron emitting radionuclide is selected from the group consisting of ¹¹C, ¹³N, ¹⁵O, ¹⁸F, ³⁸K, ⁴⁵Ti, ⁵¹Mn, ⁵²Mn, ⁵²Fe, ⁵⁵Co, ⁶⁰Cu, ⁶¹Cu, ⁶⁴Cu, ⁶⁶Ga, ⁷²As, ^{82m}Rb, ⁸³Sr, ⁸⁶Y, and ⁸⁹Zr.

3. An imaging agent for use as in claim 1 or 2, wherein the PABA metabolite is selected from the group consisting of N-acetyl-PABA, p-aminohippuric acid, and p-acetylaminohippuric acid.

4. An imaging agent for use in functional renal imaging of one or both kidneys in a subject in need thereof, characterized in that the imaging agent comprises a compound selected from the group consisting of: 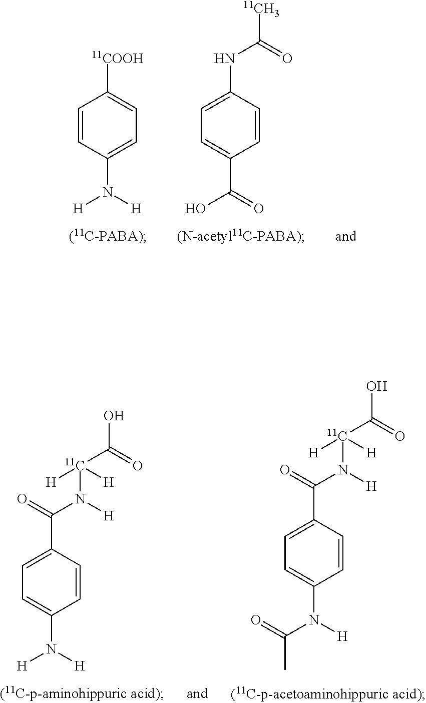
and wherein the imaging agent is administered to the subject in conjunction with obtaining a series of images at regular intervals of at least one or more regions of interest of the kidney of the subject for a period of time, and quantifying the amount of radioactivity in each interval during said time period.

5. An imaging agent for use in functional renal imaging of one or both kidneys in a subject in need thereof, characterized in that the imaging agent comprises para-amino benzoic acid (PABA) or a metabolite or derivative thereof, labeled with a positron emitting radionuclide, and wherein the imaging agent is administered to the subject in conjunction with imaging the kidney of the subject with a positron emission detecting apparatus or PET or SPECT device at regular intervals of at least one or more regions of interest of the kidney of the subject for a period of time, quantifying the amount of radioactivity in each interval during said time period, collecting the quantifying data on a computer or data storage device; and identifying a ROI and analyzing the data of the images collected.

6. An imaging agent for use as in claim 5, wherein the analyzed images of the subject are compared with analyzed images of a control kidney or kidneys.

7. An imaging agent for use as in any of claims 5-6, wherein the data of the images collected is graphed as time-activity curve.

8. An imaging agent for use as in any of claims 5-7, wherein the imaging agent is administered to the subject along with the administration to the subject of a second positron labeled imaging agent.

9. An imaging agent for use as in any of claims 5-8, wherein the imaging agent is administered to the subject along with a pharmacological agent prior to or concurrently with the imaging agent.

10. An imaging agent for use as in claim 9, wherein the pharmacological agent is selected from the group consisting of: Captopril, Enalapril, Aspirin, ARA II (valsartan) and other angiotensin II inhibitors, and furosemide and other diuretics.
